SEQUENCE LISTING (1 sequence)

SEQ ID NO: 1 - DNA, 24 bp, Artificial Sequence
  Synthetic CpG-ODN
tcgtcgttttgtcgttttgtcgtt

(19) United States
(12) Patent Application Publication
(10) Pub. No.: US 2020/0270294 A1
(43) Pub. Date: Aug. 27, 2020

(54) IMMUNOSTIMULATORY ADJUVANTS AND USES THEREOF

(71) Applicants: TURUN YLIOPISTO, Turun yliopisto (FI); ÅBO AKADEMI, Åbo (FI)

(72) Inventors: Jani Rahkila, Turku (FI); Reko Leino, Kaarina (FI); Johannes Savolainen, Turun Yliopisto (FI)

(73) Assignees: TURUN YLIOPISTO, Turun yliopisto (FI); ÅBO AKADEMI, Åbo (FI)

(21) Appl. No.: 16/063,531

(22) PCT Filed: Dec. 21, 2016
(86) PCT No.: PCT/FI2016/050908

§ 371 Date: Jun. 18, 2018

(30) Foreign Application Priority Data
Dec. 22, 2015 (FI) 20155997

(51) Int. Cl. C07H 15/26; A61K 39/39; A23L 33/10; A61P 35/00

(52) U.S. Cl. CPC C07H 15/26; A61K 39/39; A61K 2039/57; A61P 35/00; A23L 33/10

(57) ABSTRACT

The present invention relates to immunostimulatory compounds, combinations thereof, pharmaceutical or nutritional preparations comprising the same, as well as their use for modulating T helper (Th) and T regulatory (Treg) cell-mediated immune responses.

### FIELD OF THE INVENTION

The invention relates to immunostimulatory compounds, and to their use for modulating T helper (Th) and T regulatory (Treg) cell-mediated immune responses.

### BACKGROUND OF THE INVENTION

Immune responses are modulated by so called helper T cells, which can be further divided into Th1, Th2 and regulatory Treg cells based on the cytokines they excrete. Th1 cells produce for example interferon-γ (IFN-γ) and tumor necrosis factor α (TNF-α), activating macrophages to kill microbes absorbed by phagocytosis. In addition Th1 cells activate cytotoxic T cells to kill infected cells. Th2 cells are distinctive by producing interleukins (IL) 4, 5 and 13, which are important for allergic inflammations due to their ability to activate certain immune cells, namely basophils, mast cells and eosinophils. The cytokines secreted by Th1 and Th2 cells inhibit the effects of the reciprocal phenotype. Furthermore, Treg cells have the task of maintaining a balance in the immune system by secreting IL-10 which suppresses both Th1 and Th2 responses.

The IL-4 secreted by Th2 cells causes B cells to differentiate into plasma cells, which produce antibodies, but these plasma cells produce immunoglobulin E (IgE) which stimulates basophils and mast cells to secrete local mediators such as histamine and serotonin, which cause excessive mucus secretion as well as coughing, sneezing and diarrhea to get rid of the detected antigens. IL-5 on the other hand activates eosinophils which produce cytokines and chemokines that cause inflammatory responses at sites of allergen exposure.

Allergies are a type of hypersensitivity disorder, formally known as type I hypersensitivity, where the immune system reacts excessively to a normally harmless substance. In a healthy subject, allergen exposure causes T cell responses dominated by Treg and Th1 cells which cause secretion of IgG4 which removes the allergens from the body in a harmless way. In patients suffering from allergies, however, the response is strongly dominated by Th2 cells, which leads to the aforementioned inflammatory reaction.

Patients with allergies can be treated with repeated injections of increasing amounts of a specific allergen. This type of specific immunotherapy (SIT) acts to normalize a Th2-type inflammatory response towards a protective Th1 and Treg response. SIT is currently the only way of treating the underlying pathological immune response associated with allergy. It is a potent method offering long-lasting protection, but treatments take 3-5 years and the large amount of allergens injected can cause allergic reactions, and in severe cases even anaphylaxis. The treatment can, however, be optimized by using specific types of adjuvants that modify the immune response, its duration as well as increasing the production of antibodies of the correct type.

One common adjuvant used in vaccines is alum (aluminum hydroxide) which unfortunately promotes Th2-type responses which cause unfavorable IgE production. There are, however, several promising candidates such as oligodeoxynucleotides (CpG ODN), which have shown promising results in a number of Phase I-II clinical studies, and monophosphoryl lipid A (MPL) which has been approved for human use and has been shown to offer protection against allergic reactions using a significantly reduced number of injections. However, therapeutic use of these compounds is not without problems. CpG-ODN is a gene and, in addition to being expensive for large-scale synthesis, implies potential problems with the public opinion (such as gene manipulated food products). MPL adjuvant, in turn, is not a single chemical entity, but a mixture of analogues, with differences reflected in the number and length of fatty acid chains.

The immunostimulatory properties of natural β-(1→2)-linked mannosides have already been known for a long time. They have on multiple occasions been shown to stimulate the production of antibodies against Candida albicans, an opportunistic fungus that can cause severe bloodstream infections in immunocompromised persons. It is, however, carried by most humans and is, in fact, considered to be part of the normal human gut flora.

The biological activity of this class of compounds is believed to be, at least partly, due to their unique three-dimensional solution structure. In a solution they adopt a contorted α-helical conformation with 3-4 sugar units per revolution. Due to the steric clashes resulting in the unique conformation, this class of compounds is quite rare in nature, but can be found on the cell surface of several fungi of the Candida species, mainly C. albicans.

International Patent Application WO2006/096970 discloses an immunogenic conjugate comprising a plurality of oligosaccharides comprising β-D-mannopyranosyl-1→2)-β-D-mannopyranose wherein each of said oligosaccharide is linked via a linker to a protein carrier. The conjugate is useful in the preparation of a vaccine which elicits an immunogenic response, i.e. provokes acquired immunity, against Candida species, C. albicans in particular.

International Patent Application WO2007/010084 discloses immunostimulatory mannan polysaccharides comprising β-(1→2)-linked chains and β-(1→2)-D-oligomannosides, and their uses for modulating Th-mediated immune responses. However, the activity of synthesized simple β-(1→2)-linked oligomannoside chains consisting of up to four monosaccharide units was inferior as compared to natural crude oligosaccharide mixtures. Therefore, novel modifications of these synthetic oligosaccharide chains were needed.

International Patent Application WO 2012/175813 discloses β-(1→2) oligomannosides for use as modulators of Th and Treg-mediated immune responses.

There is a need in the art for further immunostimulatory compounds providing immunostimulatory properties as well as good water solubility allowing ease of formulation of the corresponding pharmaceutical and/or nutritional preparations.

### BRIEF DESCRIPTION OF THE INVENTION

An object of the present invention is to provide an immunostimulatory compound and composition for modulating T helper (Th) and T regulatory (Treg) cell-mediated immune responses.

It has been surprisingly found that an immunostimulatory compound of formula (1) provides modulation of above described Th mediated immune responses and may, thus, be used in prevention or treatment of type I atopic allergies, infectious diseases, and cancer in a subject

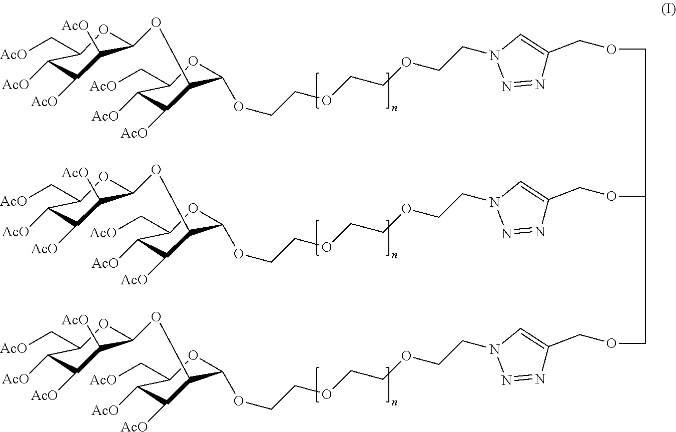

wherein each n is 0 to 2.

The compounds of the present invention bear 1) fully acetylated trivalent β-(1→2) mannobiose units, which 2) through α-linkages are connected to the central core, via 3) an ethylene glycol based linker connecting the mannobioses to the triazole groups.

Therefore, in one aspect, the present invention provides the compounds of formula (I), any combination thereof, and a composition comprising the same for modulating Th and Treg cell-mediated immune responses.

In another aspect, the present invention provides the immunostimulatory compounds of formula (I) for use as a medicament. In yet another aspect, the present invention provides the immunostimulatory compounds for use as a medicament for treating a mammal, including human, suffering from or susceptible to a condition which can be prevented or treated by inducing a Treg- and/or Th1 type, and/or inhibiting a Th2-type immune response.

Treg- and/or Th1-type immune response is induced by induction of IFN-γ production in T cells, and/or inhibition or suppression of the function of Th2-type T cells, mast cells, eosinophil granulocytes and/or basophil granulocytes. Th2-type immune response is inhibited by induction of IL-10 production in T-cells and/or inhibition or suppression of the function of Th2-type T cells, mast cells, eosinophil granulocytes and/or basophil granulocytes. The inhibition of Th2-type immune response is also based on the suppression of allergen-induced IL-4 and/or IL-5 production.

The invention also provides the immunostimulatory compounds according to the invention for use in treatment of type I immediate atopic allergy. In preferred embodiments, the type I immediate atopic allergy is selected from the group of atopic eczema/dermatitis syndrome (AEDS), allergic asthma, allergic rhinitis, allergic urticaria, food allergy, venom allergy, and allergic rhinoconjunctvitis. In further embodiments, the invention provides the immunostimulatory compound of the invention for use in treatment of infectious diseases or cancer.

A further aspect of the present invention provides the immunostimulatory compounds of formula (I) for use as an adjuvant of a vaccine.

The present invention is also directed to an immunostimulatory composition comprising one or more immunostimulatory compounds of formula (I), and a pharmaceutically acceptable carrier.

In a still further aspect, the present invention provides the immunostimulatory compounds of formula (I) for use as a food additive.

The present invention also provides a method for inducing of Treg- and/or Th1-type immune response comprising administering to a subject the composition of the invention in an amount effective to induce a Treg- and/or Th1-type immune response, and a method for inhibition of Th2-type immune response comprising administering to a subject the composition of the invention or the food of the invention in an amount effective to partially or completely inhibit development of Th2-type immune response.

Other objects, aspects, details, advantages, and specific embodiments of the present invention will become apparent from the following drawings, detailed description, examples, and dependent claims.

### BRIEF DESCRIPTION OF THE DRAWINGS

In the following the invention will be described in greater detail by means of preferred embodiments with reference to the attached drawings, in which

FIG. 1 illustrates IL-4 responses of the PBMCs stimulated with Bet v with and without compound 1, 2, 3, MPL, and CpG-ODN.

FIG. 2 illustrates TNF responses of the PBMCs stimulated with Bet v with and without compound 1, 2, 3, MPL, and CpG-ODN.

FIG. 3 demonstrates the efficacy of compound 3 in suppression of melanoma tumor growth in mice.

FIG. 4 shows ROESY spectrum of reference compound 1.

FIG. 5 illustrates the most populated conformations of reference compound 1.

FIG. 6 shows ROESY spectrum of reference compound 2.

FIG. 7 illustrates the most populated conformation of reference compound 2.

FIG. 8 shows ROESY spectrum of compound 3, with the most important correlations.

FIG. 9 illustrates the most populated conformation of compound 3.

### DETAILED DESCRIPTION OF THE INVENTION

The present invention provides a synthetic immunostimulatory fully acetylated trivalent β-(1→2) manno-oligosaccharide of formula (I), compositions comprising the same, and uses thereof for modulating helper T cell (Th)-mediated immune responses, as well as uses in the manufacture of a medicament, or a pharmaceutical or a nutritional preparation for prevention or treatment of type I atopic allergies, infectious diseases, or cancer in a subject.

Allergic inflammation is characterized by IgE antibody production, mast cell degranulation and eosinophilic inflammation. These responses are mediated by allergen-specific Th2-type immune cells that secrete cytokines such as IL-4 and IL-5. The other type of helper T cells, i.e. Th1-type immune cells, in turn, secrete cytokines such as IFN-γ and are involved in suppression of allergen-induced Th2-type immune responses. In addition, regulatory cytokine IL-10 is important in down-regulation of Th2-type immune responses.

The compounds of formula (I) are capable of modulating the above-described Th-mediated immune responses. Said modulation may occur at least by suppression of IL-4 production in human white blood cells as is demonstrated in Examples below. Owing to this activity, the compound of formula (I) is a potent inhibitor of Th2-mediated immune responses and, thus, a potent adjuvant for use in prevention or treatment of type I atopic allergies, infectious diseases, and cancer in a subject.

The compounds of formula (I) bear 1) fully acetylated trivalent β-(1→2) mannobiose units, which 2) through α-linkages are connected to the central core, via 3) a ethylene glycol based linker connecting the mannobioses to the triazole groups. The synthesis of the compound of formula (I) is preferably based on the use of a click chemistry protocol. The particular choice of the combination of the units of the compounds of formula (I) provides a sufficient water solubility.

If desired, the compounds of formula (I) may be used in any combinations in any aspects or embodiments of the present invention. As used herein, the terms “the present compound”, “the present compounds”, and any equivalents thereof, are interchangeable, and may refer to one or more compounds of formula (I) and/or to the compound of formula (II) regardless of whether the term is in singular or plural form.

Preferably, the compound of the invention bears a triethylene glycol based linker (i.e. n is 1), and is thus in accordance with formula (II)

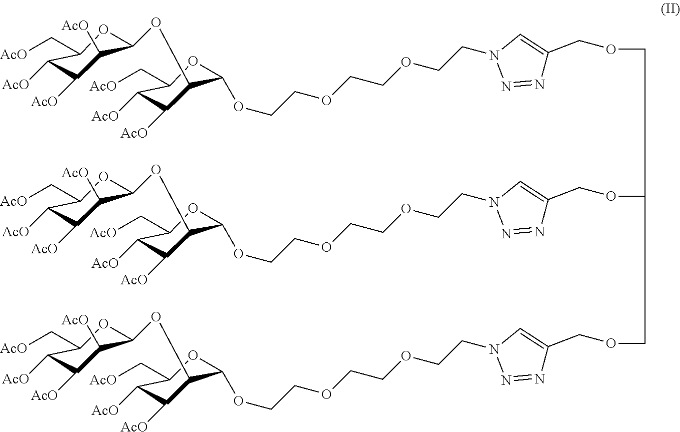

or pharmaceutically acceptable salt thereof.

The compound of formula (II) may be named as (1,2,3-tris(1-{2-[2-(2-[O-(2,3,4,6-tetra-O-acetyl-β-D-mannopyranosyl)-(1→2)-3,4,6-tri-O-acetyl-α-D-mannopyranosyloxy]-ethoxy)-ethoxy]ethyl}-4-methyloxy-1,2,3-triazolyl)propane). In the experimental section this compound is referred to as compound 3.

Despite the long linker units, which allow more flexibility and less predictable folding in three dimensional structure as compared with previously known β-(1->2) oligomannosides disclosed in WO 2012/175813, the compound of formula (I) is able to suppress Betula verrucosa-induced IL-4 response of PBMCs from persons suffering from birch allergy and is thus promising adjuvant in allergen immunotherapy. The three-dimensional structure of the compound of formula (I) appears to be highly specific and required for biological activity.

As used herein, the term “subject” refers to a mammal, preferably a human individual. Non-limiting examples of other mammalian subjects include domestic animals, farm animals, and zoo, sports, or pet animals such as dogs, cats, guinea pigs, rabbits, rats, mice, horses, cattle, cows, bears, and so on. Thus, the present compound and compositions comprising the same may be used for the medical treatment of humans, as well as for veterinary purposes.

As used herein, the term “immunostimulant” or “immunostimulatory compound” refers to a biologically active substance whose activities affect or play a role in the functioning of the host immune system by stimulating T helper type 1 and regulatory type T cell responses. Owing to their ability to intensify and modify innate immune responses and their duration, immunostimulatory compounds are, among other applications, suitable for being used as adjuvants in vaccines or SIT preparations for enhancing humoral and cellular immune responses against a vaccine or SIT immunogen co-administered together with the immunostimulatory compound.

As used herein, the term “innate immune system”, also known as “non-specific immune system” refers to one of the two distinct components of the immune system. Innate immune system consists of nonspecific defence mechanisms that are always present in a vertebrate body and ready to fight foreign antigens immediately or within hours of an antigen's appearance in the body without previous infection or vaccination. These mechanisms include physical barriers such as skin, chemicals in the blood, and immune system cells that attack foreign cells in the body. The innate immune response is activated by chemical properties of the antigen.

As used herein, the term “adaptive immune system”, also known as “acquired immune system” refers to the other distinct component of the immune system, i.e. antigen-specific immune response. The adaptive immune response is more complex than the innate, and it is required for fighting foreign antigens that have evaded or overcome the innate immune defences. The adaptive immune system is normally silent but becomes activated in the presence of said foreign antigens. First, the antigen must be processed and recognized. Once recognized, the adaptive immune system creates an army of immune cells specifically designed to attack that antigen. Adaptive immunity also includes a “memory” that makes future responses against a specific antigen more efficient. The humoral component of the adaptive immune system is mediated by antibodies produced by B lymphocytes, while the other, cell-mediated component acts through T lymphocytes.

As used herein, the term “immunogen” refers to an agent that stimulates a specific adaptive immune response against the immunogen itself. This is in contrast to immunostimulants which stimulate a non-specific activation of the immune system in order to enhance a specific immune response against a co-administered immunogen.

Hence, the present immunostimulatory compound is suitable for use as an adjuvant of a vaccine. Thus, it may be added to a vaccine as an adjuvant to stimulate the immune system's response to a target antigen, but it does not in itself confer immunity. Especially, the present compound is suitable for use as an adjuvant in injections of desensitization or allergen-specific immunotherapy. Also, the present compound may be used as an adjuvant in vaccines against infectious diseases or cancer. Preferably, when used as an adjuvant, the immunostimulatory compound according to the invention may be co-administered with the main component, the immunogen in question. However, the immunostimulatory compound according to the invention may optionally be engineered to be bonded or further linked to said immunogen or to another component of the vaccine composition.

The invention also encompasses a method for inducing a Treg- and/or Th1-type immune response. The method involves administrating to a subject the present compound or a composition thereof in an effective amount to induce the synthesis of Treg- and/or Th1-type cytokines. In preferred embodiments, the method involves, but is not limited to, the induction of IFN-γ synthesis in T cells.

The invention further involves a method for inhibiting a Th2-type immune response. The method involves administrating to a subject in need thereof, the present compound, any combination thereof, or a composition comprising the same in an effective amount to partially or completely inhibit the development of Th2-type immune response to an immunogen. In preferred embodiments, the mechanisms of inhibition include, but are not limited to, induction of IL-10 production and/or suppression of IL-4 and/or IL-5 production in T cells. The method may also involve suppression of Th2-type immune response by inhibition or suppression of the function of Th2-type T cells, mast cells and eosinophil and basophil granulocytes.

More specifically, the present immunostimulatory compound or a composition thereof is particularly suitable for use in applications wherein a Treg- and/or Th1-type immune response is induced by

a) induction of IFN-γ production in T cells, and/or

b) inhibition or suppression of the function of Th2-type T cells, mast cells, eosinophil granulocytes and/or basophil granulocytes.

Furthermore, the present immunostimulatory compound or a composition thereof is particularly suitable for use in applications wherein a Th2-type immune response is inhibited by

a) induction of IL-10 production in T cells,

b) suppression of IL-4 and/or IL-5 production in T cells, and/or

c) inhibition or suppression of the function of Th2-type T cells, mast cells, eosinophil granulocytes and/or basophil granulocytes.

The present invention also provides a method for modulating a Th-mediated immune response. Preferably, the immune response stimulated according to the invention is biased toward the Th1-type response and away from the Th2-type response. In one aspect, the method involves administrating to a subject the present compound, any combination thereof, or a composition comprising the same in an effective amount to stimulate the production of Th1-type cytokines. In preferred embodiments, the method involves, but is not limited to, the induction of IFN-γ synthesis in T cells. In other aspect, the method involves administrating to a subject the compound of formula (I), any combination thereof, or a composition comprising the same in an effective amount to partially or completely inhibit the development of Th2-type immune response to an immunogen. In preferred embodiments, the mechanisms of inhibition include, but are not limited to, induction of IL-10 production and/or suppression of IL-4 and/or IL-5 production in T cells. The method may also involve suppression of Th2-type immune response by inhibition or suppression of the function of Th2-type T cells, mast cells and eosinophil and basophil granulocytes.

The invention further relates to the use of the present compound for the manufacture of a medicament, or a pharmaceutical or nutritional preparation for prevention or treatment of type I immediate atopic allergies. In preferred embodiments, the type I immediate atopic allergy is selected from the group consisting of atopic eczema/dermatitis syndrome (AEDS), allergic asthma, allergic rhinitis, allergic urticaria, food allergy, venom allergy, and allergic rhinoconjunctivitis. In further embodiments, the compound of the invention can be used for prevention and treatment of infectious diseases caused by infectious pathogens. In still further embodiments, the compound of the invention can be used for prevention or treatment of cancer.

As used herein, the terms “prevent”, “prevention”, “preventing”, and the like refer to inhibiting completely or partially the development or onset of the disorder in a subject that has, or is at high risk of developing, said disorder.

As used herein, the terms “treat”, “treatment”, “treating”, and the like refer to administering to a subject in need of such treatment an effective amount of the present compound, any combination thereof, or a composition comprising the same to prevent the onset of, alleviate the symptoms of, stop the progression of, or cure the disorder.

As used herein, the term “effective amount” refers to an amount effective enough for achieving the desired therapeutic result or, at minimum, ameliorating the harmful effects of Th2 mediated events. Amounts and regimens for the administration of the present compound, any combination thereof, or composition comprising the same can be determined readily by those with ordinary skill in the clinical art of treating of type I hypersensitivity, infectious diseases, or cancer. Typically, the therapeutically effective amount varies from about 1 μg to several grams depending on the composition and especially on the mode of administration. For instance, a typical amount for parenteral administration of the present trivalent acetylated 1-(1-2) linked mannobiose is from 1 μg to 100 μg, preferably 2 μg to 30 μg, most preferably 3 μg to 10 μg, and for oral administration a typical amount may be much higher and vary from a few mg up to several grams.

In further embodiments, a medicament or pharmaceutical preparation of the invention may be administered to a subject by any route known in the art, including enteral, mucosal, parenteral and topical routes. The enteral routes include oral and any route involving absorption from the gastrointestinal tract. The mucosal routes include, but are not restricted to, oral, nasal, sublingual, buccal, pulmonary, transdermal and ocular routes. The parenteral routes include, but are not restricted to, intravenous, intradermal, intramuscular, and subcutaneous routes.

In some embodiments, the present compound, any combination therefor, or a composition comprising the same may be used in conjugation with a pharmaceutically acceptable carrier. An immunostimulatory composition according to the invention typically includes at least one immunostimulatory compound of formula (I), and a pharmaceutically acceptable carrier. The term “pharmaceutically acceptable carrier” refers to a carrier substance with which the active ingredient is combined to facilitate the application to a subject and that is physiologically acceptable to the recipient. Pharmaceutically acceptable carriers are readily available in the art and, depending on the intended route of administration, may be selected from the group consisting of, but not restricted to, transdermal carriers, transmucosal carriers, oral carriers, parenteral carriers, carriers for depot formulations, and carriers for extended release formulations.

The present immunostimulatory compound, any combination thereof, or a composition comprising the same may be encapsulated, incorporated or dissolved into a matrix, which can provide extended release systemic delivery. It may optionally be adapted for providing extended delivery within a localized tissue region, for example within a site of allergic reaction, infection site, or vaccination site. Such sustained release or controlled release is intended to encompass release that occurs as the result of bio-degradation of the depot or component thereof in vivo, or as the result of metabolic transformation or dissolution releasing said immunostimulatory compound. For example in subcutaneous injections, wherein the immunostimulatory compound is used as an adjuvant together with an immunogen, it might in itself function as a depot that will leak out to the blood/surrounding tissue over time.

Alternatively, the immunostimulatory compound of the present invention may be conjugated from the core unit or a carrier directly to a lipid group which can then provide a depot preparation. Such lipid-modified or lipidated immunostimulatory compounds may be used for formation of suspensions, incorporation into emulsions, lipid membranes, lipid vesicles, liposomes and the like.

In further embodiments, a pharmaceutical composition of the invention may include another therapeutic compound. The term “therapeutic compound” as used herein is preferentially an allergy medicament, an asthma medicament, an antimicrobial agent, or a cancer medicament.

In other embodiments, a pharmaceutical composition of the invention comprises an antigen. The term “antigen” broadly includes any type of molecule (e.g. protein, peptide, polysaccharide, glycoprotein, nucleic acid, or combination thereof) that is recognized by a host immune system and is capable of eliciting a specific immune response. The antigen used in the compositions of the present invention for stimulating an immune response directed to that antigen may be a synthetic, naturally-occurring or isolated molecule or a fragment thereof, and may comprise single or multiple epitopes. Thus, the compositions of the present invention may stimulate immune responses directed to single or multiple epitopes of one or more antigens. When referring to the compositions of the present invention, the terms “antigen” and “immunogen” may be used interchangeably.

In some embodiments the antigen is an allergen preparation for specific allergen immunotherapy (allergen vaccination or sublingual immunotherapy). As used herein, the term “allergen”, refers to a substance that can induce an allergic or asthmatic response in a susceptible subject, and includes but is not limited to pollens, insect venoms, animal dander, fungal spores and house dust mite. As used herein, the term “specific allergen immunotherapy”, also known as allergen immunotherapy, hyposensitization therapy or immunologic desensitization, refers to treatment of a subject with an allergic disorder by administrating gradually increasing amounts of allergen by any of the known routes to induce tolerization to the allergen to prevent further allergic reactions. Hence, the composition of the invention may also comprise an allergen preparation for specific allergen immunotherapy, and/or an additional allergy or asthma medicament.

Alternatively, a pharmaceutical composition of the invention may further comprise a microbe-specific antigen preparation for vaccination or immunization against infectious diseases and/or an antimicrobial agent. The term “infectious disease” refers to a disease arising from the presence of foreign microorganisms or infectious pathogens in the body. The term “infectious pathogens”, i.e. microbes, refers to viruses, bacteria, and parasites. As such, the term infectious pathogens also includes normal flora which is not desirable. In one aspect, combined administration of a pharmaceutical composition of the invention and a microbial antigen is useful for stimulating enhanced immune response to pathogens. The term “microbial antigens” as used herein includes intact microorganisms, as well as natural isolates and fragments or derivates thereof, and also synthetic compounds, which are identical to or similar to natural microbial antigens. In yet other embodiments, a pharmaceutical composition of the invention may comprise antibodies or antibody fragments which specifically bind or recognize microbial antigens.

In some further embodiments, a pharmaceutical composition of the invention may comprise a cancer antigen for eliciting a specific immune response against cancer cells expressing the antigen. As used herein, the terms “cancer antigen” and “tumor antigen” are interchangeable and they refer to a compound, such as a peptide, expressed by a cancer cell or a tumor cell and which is capable of provoking an immune response. More specifically, “tumor-specific antigens” are antigens that are specifically associated with tumor cells but not with normal cells. Non-limiting examples of tumor-specific antigens are those encoded by mutant cellular genes, such as oncogenes, suppressor genes, and fusion proteins resulting from internal deletions or chromosomal translocations. “Tumor-associated antigens” are present in both tumor cells and normal cells but are present in a different quantity or a different form in tumor cells. Still other cancer antigens are encoded by viral genes such as those carried on RNA and DNA viruses. The differential expression of cancer antigens in normal and cancer cells can be exploited in order to target cancer cells.

In some embodiments, cancers to be treated by the present methods, compounds, and compositions include, but are not limited to, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, and combinations thereof. A preferred cancer type to be treated in accordance with the present invention is melanoma.

Cancer antigens specific for or associated with different cancers are well known in the art. Therefore, a skilled person art can easily select a cancer antigen to be comprised in a composition of the present invention depending on the cancer type to be treated. Cancer antigens can be prepared by methods well known in the art. For example, these antigens can be prepared from cancer cells either by preparing crude extracts of cancer cells, by partially purifying the antigens, by recombinant technology, or by de novo synthesis of known antigens. Further, the antigen may be a complete antigen, or it may be a fragment of a complete antigen comprising at least one epitope.

In yet other embodiments, the compound of the invention may be used as a food additive. The compound of the invention may also be a nutritional preparation. The nutritional preparation is preferentially enterally administrable, e.g. a powder, a tablet, a capsule, a liquid concentrate, a solid product, or a ready-to drink beverage. Alternatively, the nutritional preparation is combined with a matrix suitable as an additive of usual food products. In other embodiments, the nutritional preparation of the invention is used for enrichment of infant formulas or other functional food products. A further embodiment comprising the composition of the invention could be a chewing gum.

### Examples

### General

All reagents for synthetic work were purchased from Sigma-Aldrich, were of at least reagent grade and used without further purification. Dry CH₂Cl₂ was obtained by distillation over CaH₂ and dry DMF was purchased as such and stored over molecular sieves. TLC was performed on aluminum sheets precoated with silica gel 60 F₂₅₄ (Merck) and the spots were visualized by UV and charring by treatment with H₂SO₄ in MeOH (20% v/v) followed by heating. Column chromatography was performed using Silica gel 60 (0.040-0.060 mm Merck). Optical rotations were measured with a Perkin-Elmer 241 polarimeter using the D-line of sodium at 589 nm. HRMS were recorded on a Bruker MicroToF-Q with electrospray ionization operating in positive mode. NMR spectra were recorded on Bruker Avance spectrometers operating at either 600.13 Hz or 500.13 Hz (¹H) and 150.90 Hz or 125.77 Hz (¹³C). The complete assignment of proton and carbon spectra was carried out by recording a standard set of NMR experiments, ¹H NMR, ¹³C NMR, DQF-COSY, HSQC (both coupled and decoupled) and HMBC. The complexities of the spectra were reduced to monosaccharide level by using 1D-TOCSY. ROESY and DOSY methods were used to help with the conformational studies. The chemical shifts are referenced to an internal standard (tetramethylsilane, δ=0.0 ppm in both ¹H and ¹³C) or residual solvent signals (CDCl₃, δ=7.26 ppm in ¹H and 77.16 ppm in ¹³C) and reported with two decimals for ¹H NMR and one decimal for ¹³C NMR. Where this is not enough to separate the signals, an additional decimal is reported. Accurate coupling constants were, where possible, determined with the NMR simulation software PERCH and reported with one decimal. To avoid unnecessary bloating of the NMR data, each coupling constant is reported only the first time it is encountered.

The molecular modeling and visualization was done in Maestro and the calculations were performed by Desmond using the built-in interface in Maestro. Initially the molecules were constructed in Maestro after which the calculations were carried out using Desmond controlled via the built-in interface in Maestro. To get a good starting point for the molecular dynamics simulations a quick minimization sequence over 1000 steps was performed after constructing the molecules and the results visually inspected in order to see if they were reasonable. The molecular dynamics simulations started with a simulated annealing sequence where the temperature was first raised to 600 K and then slowly lowered from to the final simulation temperature, which in our case was lower than ambient temperature in order to shift the solvent signals so they do not overlap with important signals in the NMR spectra. Furthermore the lower temperature caused some of the overlapping proton signals to shift apart making it easier to extract information from the ROESY spectra. The annealing was repeated to make sure that the molecules were not stuck in a high energy local minimum. After the annealing, the molecular dynamics simulation was run for 2 ns during which the structures were stored every 1 ps. From these structures the average distances between protons were measured, and since the NOE effect is proportional to 1/r⁶, this factor was used as weight when calculating the average distances. Out of the generated structures, the most populated conformation families were analysed and verified using the correlations seen in the recorded ROESY spectra.

### Experimental Synthesis of Compound of Formula (1)

Compounds of formula (I) may be synthesized for example by following the below reaction scheme:

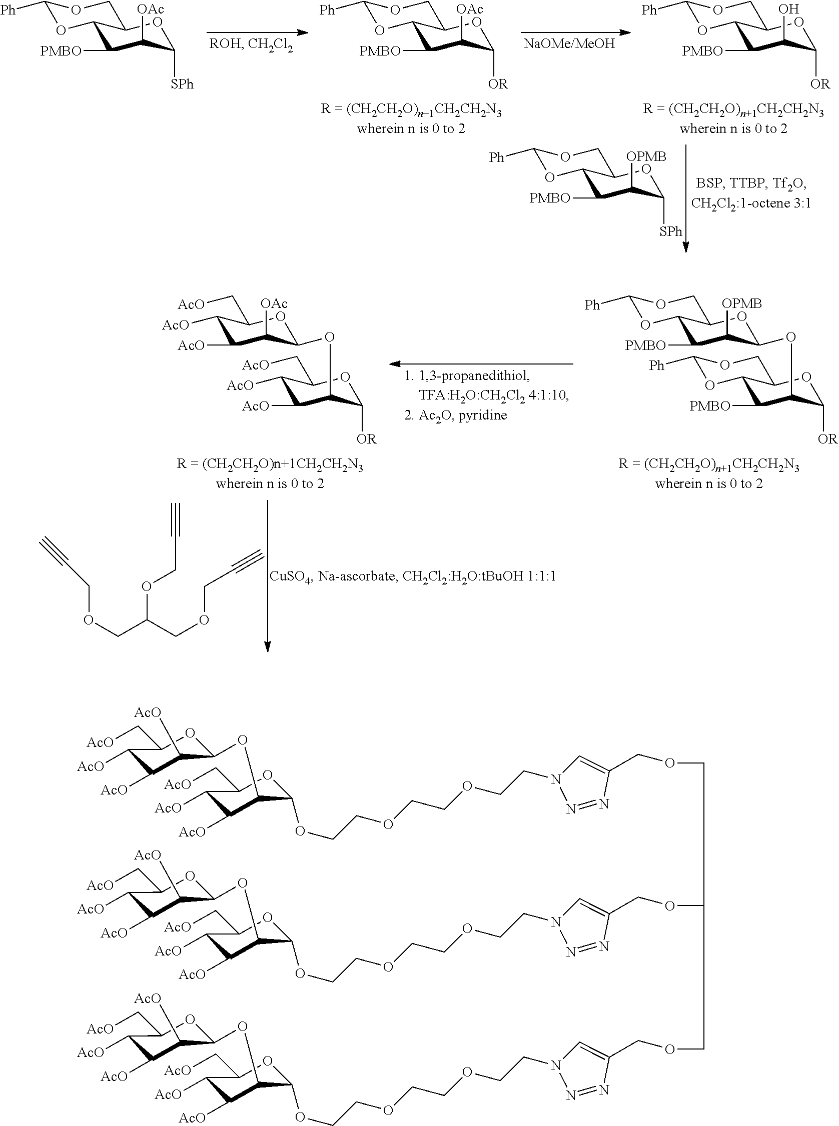

Compound of formula (II) may be synthesized for example by following the below reaction scheme:

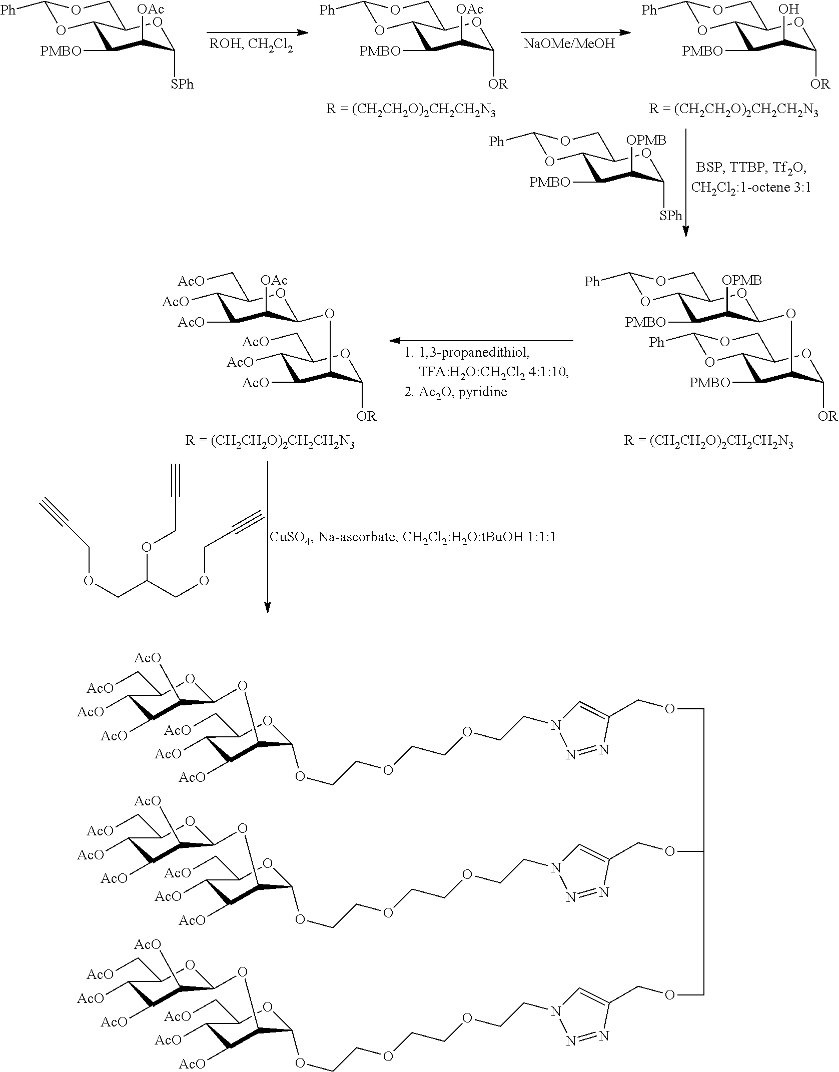

### 2-[2-(2-azidoethoxy)ethoxy]ethyl 2-O-acetyl-4,6-O-benzylidene-3-O-(4-methoxybenzyl)-α-D-mannopyranoside (5)

To a solution of phenyl 2-acetyl-4,6-O-benzylidene-3-O-(4-methoxybenzyl)-thio-α-D-mannopyranoside (1000 mg, 1.92 mmol, 1 equivalent) and 2-[2-(2-azidoethoxy)ethoxy]ethanol (403 mg, 2.30 mmol, 1.2 equivalents) in dry CH₂Cl₂ (40 ml) at −40° C. were added 4 Å molecular sieves NIS (517 mg, 2.30 mmol, 1.2 equivalents) and TMSOTf (83 μl, 0.23 mmol, 0.24 equivalents). The reaction mixture was stirred at −40° C. for 2 h and then quenched by adding a satd. solution of NaHCO₃ (20 ml). The reaction mixture was brought to room temperature and diluted with CH₂C₂ (50 ml) and washed with satd. NaHCO₃ solution (50 ml) after which the aqueous layer was extracted with CH2Cl2 (2×50 ml). The combined organic layers were washed with of brine (100 ml), dried over Na₂SO₄ and concentrated. The crude mixture was purified by column chromatography (hexane:EtOAc 2:1→1:1) to afford the pure product 5 as a slightly yellow oil. Rf: 0.33 (hexane: EtOAc 1:1). Yield 790 mg (70%).

[α]_{D}²⁴=+20.0° (c 2.30, CHCl₃).

¹H NMR (600.13 MHz, CDCl₃, 25° C.): δ=7.50 (m, 2H, arom. H), 7.40-7.34 (m, 3H, arom. H), 7.27 (m, 2H, arom. H), 6.83 (m, 2H, arom. H), 5.62 (s, 1H, 4,6-OCHPh), 5.42, (dd, 1 H, JH-2, H-1=1.6 Hz, JH-2, H-3=3.5 Hz, H-2), 4.83, (d, 1 H, H-1), 4.64 and 4.58 (each d, each 1 H, J=−11.56 Hz, 3-OCH2Ph), 4.25 (dd, 1H, JH-6a, H-5=4.8 Hz, JH-6a, H-6b=−10.24 Hz, H-6a), 4.03 (dd, 1H, JH-4, H-3=10.0 Hz, JH-4, H-5=9.5 Hz, H-4), 4.01 (dd, 1H, H-3), 3.91 (ddd, 1H, JH-5, H-6b=10.4 Hz, H-5), 3.83 (dd, 1H, H-6b), 3.80 (m, 1H, H-1′a), 3.79 (s, 3H, 3-OCH₃), 3.68-3.61 (m, 9H, H-1′b, H-2′, H-3′, H-4′, H-5′), 3.34 (m, 2H, H-6′), 2.15 (s, 3H, COCH₃).

¹³C NMR (150.9 MHz, CDCl₃, 25° C.): δ=170.2 (2-OCOCH₃), 159.2, 137.5, 130.1, 129.3, 128.9, 128.1, 126.1, 113.7 (arom. C), 101.5 (4,6-OCHPh), 98-8 (C-1), 78.3 (C-4) 73.6 (C-3), 71.8 (3-OCH₂Ph), 70.8, 70.7, 70.1 (C-2′, C-3′, C-4′, C-5′), 69.7 (C-2), 68.7 (C-6), 67.0 (C-1′), 63.8 (C-5), 55.2 (3-OCH₃), 50.6 (C-6′), 21.0 (2-OCOCH₃).

HRSM: m/z calcd. for C₂₉H₄₁N₄O₁₀ [M+NH₄]⁺: 605.2817, found: 605.2829, m/z calcd. for C₂₉H₃₇N₃NaO₁₀ [M+Na]⁺: 610.2371, found: 610.2361.

### 2-[2-(2-azidoethoxy)ethoxy]ethyl 4,6-O-benzylidene-3-O-(4-methoxybenzyl)-α-D-mannopyranoside (7)

The pH of a solution of 5 (490 mg, 0.83 mmol) in dry methanol (4 ml) under argon atmosphere was adjusted to ˜10-12 by adding a few drops of a 5.4 M solution of NaOMe in MeOH. The reaction mixture was stirred at room temperature for 30 min and then neutralized with DOWEX-50WX8 H⁺ form. The reaction mixture was filtered and concentrated to afford the pure product 7 as a yellow/orange oil. Yield: 441 mg (97%).

[α]_{D}²⁴=+21.0° (c 1.10, CHCl₃).

¹H NMR (600.13 MHz, CDCl₃, 25° C.): δ=7.51 (m, 2H, arom. H), 7.42-7.35 (m, 3H, arom. H), 7.30 (m, 2H, Arom. H), 6.88 (m, 2H, arom. H), 5.61 (s, 1H, 4,6-OCHPh), 4.91 (d, 1H, JH-1, H-2=1.5 Hz, H-1), 4.78 and 4.65 (each d, each 1 H, J=−11.4 Hz, 3-OCH₂Ph), 4.26 (dd, 1H, JH-6a, H-5=4.5 Hz, JH-6a, H-6b=−9.3 Hz, H-6a), 4.081 (dd, 1 H, JH-2, H-3=3.5 Hz, H-2), 4.076 (dd, 1 H, JH-4, H-3=9.6 Hz, JH-4, H-5=9.5 Hz, H-4), 3.93 (dd, 1H, H-3), 3.88 (ddd, 1H, JH-5, H-6b=9.6 Hz, H-5), 3.84 (dd, 1H, H-6b), 3.85-3.82 (m, 1H, H-1′a), 3.81 (s, 3H, 3-OCH3), 3.71-3.65 (m, 9H, H-1′b, H-2′, H-3′, H-4′, H-5′), 3.37 (m, 2H, H-6′).

¹³C NMR (150.9 MHz, CDCl₃, 25° C.): δ=159.5, 137.7, 130.3, 129.6, 129.0, 128.3, 126.2, 114.0 (arom. C), 101.6 (4,6-OCHPh), 101.1 (C-1), 79.0 (C-4), 75.4 (C-3), 72.8 (3-OCH2Ph), 70.91, 70.87, 70.4, 70.3 (C-2′, C-3′, C-4′, C-5′), 70.0 (C-2), 69.0 (C-6), 66.9 (C-1′), 63.4 (C-5), 55.4 (3-OCH₃), 50.8 (C-6′).

HRSM: m/z calcd. for C₂₇H₃₉N₄O₉ [M+NH₄]⁺: 563.2712, found: 563.2691, m/z calcd. for C₂₇H₃₅N₃NaO₉ [M+Na]⁺: 568.2266, found: 568.2267.

### 2-[2-(2-azidoethoxy)ethoxy]ethyl O-[4,6-O-benzylidene-2,3-di-O-(4-methoxybenzyl)-β-D-mannopyranosyl]-(1-+2)-4,6-O-benzylidene-3-O-(4-methoxybenzyl)-α-D-mannopyranoside (9)

To a solution of Phenyl 4,6-O-benzylidene-2,3-di-O-(4-methoxybenzyl)-thio-α-D-mannopyranoside (1059 mg, 1.76 mmol, 1.3 equivalents) in dry CH2Cl2 (15 ml) and 1-octene (5 ml) was added 4 Å molecular sieves after which the solution was cooled down to −60° C. BSP (526 mg, 2.12 mmol, 1.56 equivalents), TTBP (568 mg, 2.71 mmol, 2 equivalents) and Tf₂O (386 μl, 2.29 mmol, 1.69 equivalents) were added and the reaction mixture was stirred at −60° C. for 30 min. The reaction mixture was then cooled down to −78° C. and 7 (740 mg, 1.36 mmol, 1 equivalent) in dry CH₂Cl₂ (5 ml) was added. The reaction mixture was stirred at −78° C. for 3 h and then quenched by adding Et₃N (1 ml) and allowing the reaction mixture return to room temperature over 30 min. The reaction mixture was diluted with CH₂Cl₂ (50 ml) and then washed with satd. NaHCO₃ solution (50 ml). The water layer was extracted with CH₂Cl₂ (2×50 ml) and the combined organic layers were washed with brine (100 ml), dried over Na₂SO₄ and concentrated. Column chromatography (Hexane: EtOAc 2:1→1:1) afforded pure 9 as a clear oil. Rf=0.35 (Hexane:EtOAc 1:1). Yield: 613 mg (43%).

[α]_{D}²⁴=−36.0° (c 1.36, CHCl₃).

¹H NMR (600.13 MHz, CDCl₃, 25° C.): δ=7.51-7.47 (m, 4H, arom. H), 7.43 (m, 2H, arom. H), 7.40-7.32 (m, 8H, arom. H), 7.19 (m, 2H, arom. H), 6.86-6.79 (m, 6H, arom. H), 5.59 (s, 1H, 4B,6B—OCHPh), 5.51 (s, 1H, 4A,6A-OCHPh), 4.96 and 4.89 (each d, each 1 H, J=−11.9 Hz, 2B—OCH2Ph), 4.84 (d, 1H, JH-1A, H-2A=0.7 Hz, H-1A), 4.71 and 4.62 (each d, each 1 H, J=−11.5 Hz, 3A-OCH₂Ph), 4.60 and 4.53 (each d, each 1 H, J=−12.0 Hz, 3B—OCH2Ph), 4.60 (s, 1H, H-1B), 4.27 (dd, 1H, JH-2A, H-3A=3.0 Hz, H-2A), 4.26 (dd, 1H, JH-6Ba, H-5B=5.9 Hz, JH-6Ba, H-6Bb=−10.6 Hz, H-6Ba), 4.25 (dd, 1H, JH-6Aa, H-5=4.9 Hz, JH-6Aa, H-6Ab=−11.2 Hz, H-6Aa), 4.22 (dd, 1H, JH-4B, H-3B=9.8 Hz, JH-4B, H-5B=9.5 Hz, H-4b), 4.08 (dd, 1H, JH-4A, H-3A=9.9 Hz, JH-4A, H-5A=9.7 Hz, H-4A), 3.96 (dd, 1H, H-3A), 3.93 (d, 1H, JH-2B, H-3B=3.1 Hz, H-2B) 3.87 (dd, 1H, JH-6Bb, H-5B=9.9 Hz, H-6Bb), 3.85 (ddd, 1H, JH-5A, H-6Ab=9.9 Hz, H-5A), 3.82 (m, 1H, H-1′), 3.80 (s, 3H, OCH3), 3.774 (s, 3 H, OCH3), 3.766 (dd, 1 H, H-6Ab), 3.76 (s, 3H, OCH3), 3.69-3.59 (m, 9H, H-1′b, H-2′, H-3′, H-4′, H-5′), 3.55 (dd, 1H, H-3B), 3.32 (m, 2H, H-6′), 3.30 (ddd, 1H, H-5B).

¹³C NMR (150.9 MHz, CDCl₃, 25° C.): δ=159.23, 159.22, 159.1, 137.71, 137.70, 131.1, 130.7, 130.52, 130.45, 129.3, 129.2, 128.9, 128.30, 128.26, 126.22, 126.15, 113.79, 113.65, 113.6 (arom. C), 101.7 (4A,6A-OCHPh), 101.5 (4B,6B—OCHPh), 101.0 (C-1B), 98.5 (C-1A), 78.7 (C-4A), 78.6 (C-4B), 77.3 (C-3B), 75.3 (C-2B), 74.9 (C-2A), 74.2 (2B—OCH₂Ph), 72.0 (3B—OCH₂Ph), 70.94 (3A-OCH₂Ph), 70.91, 70.8, 70.3, 70.2 (C-2′, C-3′, C-4′, C-5′), 69.0 (C-6A), 68.7 (C-6B), 67.9 (C-5B), 67.0 (C-1′), 64.3 (C-5A), 55.38, 55.36, 55.3 (3×OCH₃), 50.7 (C-6′).

HRSM: m/z calcd. for C₅₆H₆₉N₄O₁₆ [M+NH4]⁺: 1053.4703, found: 1053.4669, m/z calcd. for C₅₆H₆₅N₃NaO₁₆ [M+Na]⁺: 1058.4257, found: 1058.4208.

### 2-[2-(2-azidoethoxy)ethoxy]ethyl 0-(2,3,4,6-tetra-O-acetyl-β-d-mannopyranosyl)-(1→2)-3,4,6-tri-O-acetyl-α-D-mannopyranoside (11)

To a solution of 9 (200 mg, 0.19 mmol, 1 equivalent) in 10 ml of CH₂Cl₂ was added 1,3-propanedithiol (155 μl, 1.54 mmol, 8 equivalents) and the mixture was cooled down on an ice bath. TFA (4 ml) and H2O (1 ml) were added and the reaction mixture was stirred at room temperature for 3 h. The reaction mixture was then diluted with H₂O (100 ml) and washed with CH₂Cl₂ (4×50 ml) after which the aqueous layer was evaporated and co-evaporated with toluene. The residue was dissolved in pyridine (20 ml) and cooled on an ice bath while Ac20 (10 ml) was added. The reaction mixture was then stirred at room temperature for 18 h after which the reaction was cooled on an ice bath and quenched by adding methanol (10 ml). The reaction mixture was diluted with CH₂C₂ (50 ml), washed with H₂O (4×50 ml) and brine (50 ml). The organic layer was dried over Na₂SO₄ and concentrated. The crude mixture was purified by column chromatography (hexane: EtOAc 1:1→CH₂Cl₂: methanol 20:1) to afford pure 11 as a clear oil. Rf=0.34 (CH2Cl₂: methanol 20:1). Yield: 113 mg (75%).

[α]_{D}²⁴=−51.5° (c 1.62, CHCl₃).

¹H NMR (600.13 MHz, CDCl₃, 25° C.): δ=5.51 (dd, 1H, JH-2B, H-1B=0.7 Hz, JH-2B, H-3B=3.4 Hz, H-2B), 5.27 (t, 1H, JH-4A, H-3A=JH-4A, H-5A=10.1 Hz, H-4A), 5.22 (dd, 1H, JH-4B, H-3B=10.0 Hz, JH-4B, H-5B=9.9 Hz, H-4B), 5.05 (dd, 1H, H-3B), 5.02 (dd, 1H, JH-3A, H-2A=3.4 Hz, H-3A), 4.88 (d, 1H, JH-1A, H-2A=1.8 Hz, H-1A), 4.69 (d, 1H, H-1B), 4.36 (dd, 1H, H-2A), 4.32 (dd, 1H, JH-6Bb, H-5B=6.0 Hz, JH-6Bb, H-6Ba=−12.2 Hz, H-6Bb), 4.26 (dd, 1H, JH-6Ab, H-5A=3.8 Hz, JH-6Ab, H-6Aa=−12.3 Hz, H-6Ab) 4.06 (dd, 1H, JH-6Aa, H-5A=2.3 Hz, H-6Aa), 4.02 (dd, 1H, JH-6Ba, H-5B=2.4 Hz, H-6Ba), 3.94 (ddd, 1H, H-5A), 3.81 (m, 1H, H-1′a), 3.72-3.64 (m, 9H, H-1′b, H-2′, H-3′, H-4′, H-5′), 3.64 (ddd, 1H, H-5B), 3.40 (m, 2H, H-6′), 2.25 (s, 3H, 2B—COCH₃), 2.13 (s, 3H, 6A-COCH₃), 2.10 (s, 3H, 6B—COCH₃), 2.05 (s, 3H, 4A-COCH₃), 2.03 (s, 3H, 4B—COCH₃), 2.02 (s, 3H, 3A-COCH₃), 2.01 (s, 3H, 3B—COCH₃).

¹³C NMR (150.9 MHz, CDCl₃, 25° C.): δ=171.0 (6A-COCH₃), 170.7 (6B—COCH₃), 170.3 (2B—COCH₃), 170.2 (3A-COCH₃), 170.0 (3B—COCH₃), 169.7 (4B—COCH₃), 169.3 (4A-COCH₃), 97.5 (C-1A), 96.3 (C-1B), 72.22 (C-5B), 72.17 (C-2A), 70.73, 70.72 (C-3′, C-4′), 70.6 (C-3B), 70.3 (C-3A), 70.2 (C-2′), 70.1 (C-5′), 68.52 (C-2B), 68.50 (C-5A), 67.1 (C-1′), 66.1 (C-4B), 65.1 (C-4A), 62.5 (C-6B), 61.8 (C-6A), 50.7 (C-6′), 20.8, 20.74, 20.70, 20.62, 20.57 (COCH₃).

HRSM: m/z calcd. for C₃₂H₅₁N₄O₂₀ [M+NH₄]⁺: 811.3091, found: 811.3083, m/z calcd. for C₃₂H₄₇N₃NaO₂₀ [M+Na]⁺: 816.2645, found: 816.2621.

### 1,2,3-tris (1-{2-[2-(2-[O-(2,3,4,6-tetra-O-acetyl-β-D-mannopyranosyl)-(1→2)-3,4,6-tri-O-acetyl-α-D-mannopyranosyloxy]ethoxy)ethoxy]ethyl}-4-methyloxy-1,2,3-triazolyl)propane (3)

To a solution of 11 (70 mg, 0.088 mmol, 3.3 equivalents) and 1,2,3-tris (prop-2-yn-1-yloxy)propane (5.5 mg, 0.027 mmol, e equivalent) in of CH₂Cl₂ (2 ml) were added t-BuOH (2 ml) and H₂O (2 ml). CuSO₄ (2.8 mg, 0.017 mmol, 0.66 equivalents) and Na-ascorbate (7.0 mg, 0.035 mmol, 1.32 equivalents) were added and the reaction mixture was stirred at 55° C. for 18 h. A saturated solution of NH₄Cl (10 ml) and H₂O (10 ml) were added and the reaction mixture was extracted with CH2Cl2 (4×20 ml). The combined organic layers were dried over Na₂SO₄ and concentrated. The crude mixture was purified by column chromatography (CH₂Cl₂: methanol 20:1→5:1) to afford the pure product 3 as a white solid. Rf=0.16 (CH₂Cl₂: methanol 20:1). Yield: 62 mg (89%).

[α]_{D}²⁴=−37.0° (c 1.05, CHCl₃).

¹H NMR (600.13 MHz, CDCl₃, 25° C.): δ=7.81, 7.772, 7.769 (each s, each 1 H, 3×triaz. H), 5.52-5.49 (m, 3H, 3×H-2B), 5.27 (m, 3H, 3×H-4A), 5.23 (m, 3H, 3×H-4B), 5.06 (m, 3H, 3×H-3B), 5.01-4.97 (m, 3H, 3×H-3A), 4.85 (m, 3H, 3×H-1A), 4.78 (s, 2H, G2-OCH2), 4.72 (m, 3H, 3×H-1B), 4.64 (s, 4H, G1-OCH-2), 4.60-4.51 (m, 6H, 6×H-6′), 4.36-4.30 (m, 6H, 3×H-6Bb, H-2A), 4.24 (m, 3H, 3×H-6Ab), 4.06 (m, 3H, 3×H-6Aa), 4.03-3.98 (m, 3H, 3×H-6Ba), 3.95-3.86 (m, 9H, 3×H-5A, 6×H-5′), 3.85-3.75 (m, 4H, H-G2, 3×H-1′a), 3.68-3.58 (m, 28H, 3×H-5B, 4×H-G1, 3×H-1′b, 6×H-2′, 6×H-3′, 6×H-4′), 2.24, 2.12, 2.09, 2.04, 2.03, 2.02, 2.01 (each s, each 9 H, 21×COCH₃).

¹³C NMR (150.9 MHz, CDCl₃, 25° C.): δ=171.0, 170.6, 170.33, 170.29, 170.0, 169.7, 169.3 (COCH₃), 145.1, 144.7 (C-4, triaz), 124.0, 123.9 (C-5, triaz), 97.6 (C-1A), 96.29, 96.27 (C-1B), 77.2 (C-G2), 72.11, 72.08 (C-5B, C-2A), 70.67, 70.65 (C-3B), 70.51, 70.49 (C-3′, C-4′),70.29, 70.26 (C-3A), 70.2 (C-G1), 70.1 (C-2′), 69.5 (C-5′), 68.53 (C-2B), 68.49 (C-5A), 67.0 (C-1′), 66.1 (C-4B), 65.1 (C-4A), 64.8 (G1-OCH₂), 63.8 (G2-OCH₂), 62.4 (C-6B), 61.8 (C-6A), 50.20, 50.16 (C-6′), 20.79, 20.75, 20.7, 20.62, 20.57 (COCH₃).

HRSM: m/z calcd. for C₁₀₈H₁₅₅N₉NaO₆₃ [M+Na]+: 2608.9094, found: 2608.8944.

### Biological Studies

### Allergen-Induced Cytokine Responses

### Biological Study Subjects

During pollen season, 14 adult birch allergic subjects with allergic rhinoconjunctivitis (12 females and two males) were enrolled in the study (mean age 42.5 years, SD 12.4 years; mean birch-specific IgE (Immunocap, Thermo Fisher Scientific Phadia, Uppsala, Sweden) 36.1 kU/1, SD 31.5 kU/1). They were selected for the study from an earlier cohort based on good birch induced IL-4 responses during pollen season. All samples were taken after informed consent. The study was approved by the local ethics committee.

### Adjuvants in the Biological Studies

Compound 1 (1,2,3-tris[1-(3-{0-(2,3,4,6-tetra-O-acetyl-β-D-mannopyranosyl)-(1→2)-3,4,6-tri-O-acetyl-α-D-mannopyranosyloxy}ethyl)-4-methyloxy-1,2,3-triazolyl]propane) and compound 2 (1,2,3-tris[1-(3-{O-(2,3,4,6-tetra-O-acetyl-β-D-mannopyranosyl)-(1-+2)-3,4,6-tri-O-acetyl-α-D-mannopyranosyloxy}propyl)-4-methyloxy-1,2,3-triazolyl]propane) were synthesized as previously described in WO 2012/175813. Compound 3 (1,2,3-tris(1-{2-[2-(2-[O-(2,3,4,6-tetra-O-acetyl-β-D-mannopyranosyl)-(1→2)-3,4,6-tri-O-acetyl-α-D-mannopyranosyloxy]ethoxy)-ethoxy]ethyl}-4-methyloxy-1,2,3-triazolyl)propane) was prepared according to the methods described herein. Synthetic MPL and CpG-ODN (tlrl-2006) were purchased from Invivogen (San Diego, Calif., USA). The CpG-ODN had a sequence of 5′-TCG TCG TTT TGT CGT TTT GTC GTT-3′ (SEQ ID NO: 1), identical to one used in a previous study.

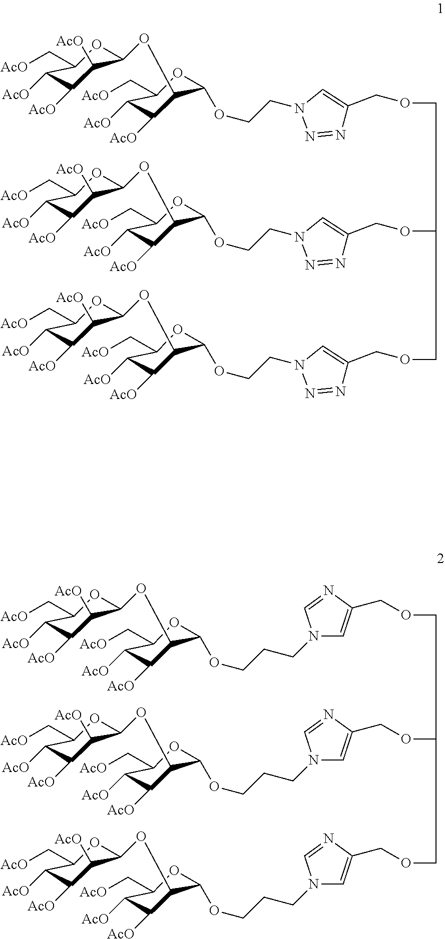

### PBMC Cultures

Peripheral blood mononuclear cells (PBMC) were isolated by Ficoll-Paque density gradient centrifugation (Ficoll-Paque PLUS, GE Healthcare BioSciences AB, Uppsala, Sweden) from heparinized blood samples from study subjects during pollen season. The PBMC were washed twice with Hanks' balanced salt solution (HBSS) buffered with NaHCO₃ (pH 7.4) and resuspended in RPMI-1640 culture medium (Invitrogen Co., Carlsbad, Calif., USA) supplemented with 5% autologous serum, 2.5 mM L-glutamine (Sigma-Aldrich Co., St. Louis, Mo.) and 100 μg/ml gentamycin sulfate (Biological Industries Ltd., Kibbutz Beit Haemek, Israel) and applied on 48-well flat-bottomed cell culture plates (Costar, Corning Inc., New York, United States) at a density of 106/ml. Cells were co-cultured in the presence of birch allergen (50 μg/ml, Betula verrucosa, Bet v, Aquagen, ALK-Abellò A/S, Horsholm, Denmark) and compounds 1, 2, 3 and MPL with concentrations 1, 10 and 100 μg/ml and adjuvant CpG-ODN with concentrations 2, 20 and 200 μg/ml to achieve equal molarities. Medium alone served as an unstimulated control. All incubations were performed at +37° C. in humidified atmosphere with 5% CO₂. Supernatants from cultures performed in duplicate were collected 72 h after beginning of the stimulation and stored at −70° C.

### IL-4 and TNF Production

The cytokines IL-4 and TNF in supernatants were measured with highsensitivity human cytokine Lincoplex kits (LINCO Research, St. Charles, Mo., USA). The assays were performed in accordance with the manufacturer's protocol by employing Luminex technology.

### Statistics

Wilcoxon's Signed Rank test was used to test statistical significance in PBMC experiments. Single mice groups were compared by the nonparametric Mann-Whitney U-test using GraphPad Prism software (v.5, GraphPad Software Inc., La Jolla, Calif., USA). Data are expressed as mean±SEM and P-values of <0.05 are considered statistically significant.

### Results

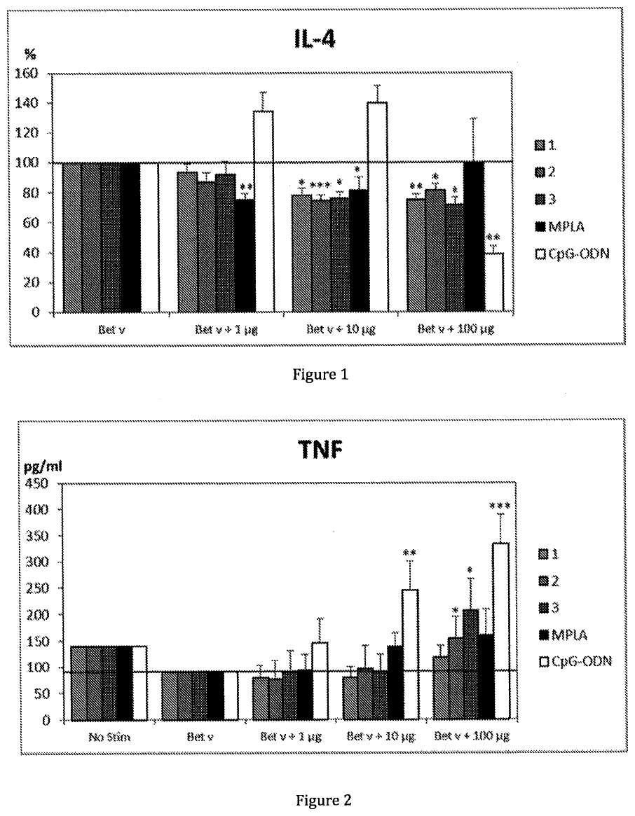

The effects of compounds 1, 2, 3, MPL and CpG-ODN on allergen (Bet v) induced cytokine responses in PBMC cultures of 14 birch allergic rhinitis patients are presented in FIG. 1. Stimulation with birch induced significant response of Th2 cytokine IL-4 (mean 45.3 pg/ml, SEM 11.2 pg/ml) as compared to nonstimulated culture (mean 0.4 pg/ml, SEM 0.2 pg/ml) (p=0.0015). A significant suppression of birch-induced production of IL-4 was seen with 10 (p<0.001) and 100 g/ml (p=0.036) of compound 2, 10 (p=0.016) and 100 l/ml (p=0.021) of compound 3, 10 (p=0.016) and 100 g/ml (p=0.006) of compound 3, 1 (p=0.002) and 10 μg/ml (P=0.0.013) of MPL and 200 μg/ml of CpG-ODN (p=0.002). Dose-response curves of suppression were seen only with compounds 1-3 as lower concentrations of CpG-ODN increased the IL-4 production and the highest concentration of MPL had no suppressive effect. Significantly increased production of pro-inflammatory cytokine TNF was seen with 100 μg/ml (p=0.021) of compound 2, 100 l/ml (p=0.0039) of compound 3 and 20 (p=0.0026) and 200 μg/ml (p<0.001) of CpG-ODN (FIG. 2).

### In Vivo Xenograft Model of Cancer Immunotherapy

### Material and Methods

3*10⁶ B16 melanoma cells in 100 μl PBS were injected s.c. into the right flank of seven-week-old female C57BL/6 mice. Compound 3 was given i.p. 50 μg/injection in 200 μl PBS five times at 3-day intervals, starting on day 0. Each treatment group contained 10 mice. Tumor volumes were calculated using the formula 0.5*length*width². Animals were sacrificed when tumor volumes reached 800 mm³. Animal experimentations were made following the procedures accepted by Eteli-Suomen Aluehallintavirasto (permission ESAVI/480/Apr. 10.2007/2016).

### Results

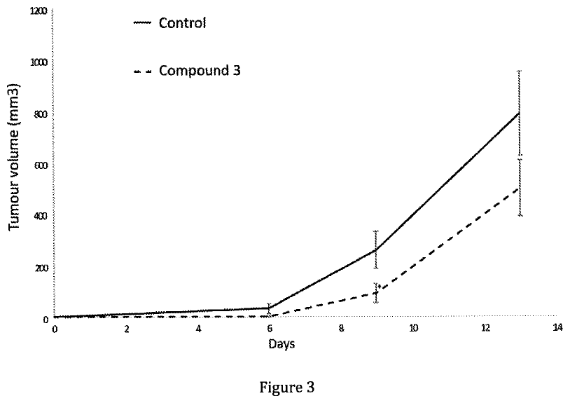

Tumor growth in mice was extremely aggressive and mice had to be sacrificed on Day 13. Tumor growth started later in mice treated with compound 3 and was slower until Day 9 as compared to control. On Day 9 the tumor volume was statistically significantly smaller in mice treated with compound 3 as compared to control mice (FIG. 3).

### Conformational Studies

In order to predict and accurately determine the effect of biologically active molecules, it is of importance to investigate their behavior in solution. Such investigations typically involve studies on the three dimensional structure of the molecules and their changes over time. Such studies are not always straightforward, with several factors including temperature and the solvent influencing the dynamic behavior of molecules. In the present study, compounds 1, 2, and 3 were studied by NMR spectroscopy, including diffusion ordered spectroscopy (DOSY) and rotating-frame overhauser effect spectroscopy (ROESY) methods.

The NOE effect can be either positive for small molecules that tumble rapidly in solution, or negative for larger molecules, such as proteins, that tumble more slowly. As a consequence of this, there is a region where the NOE effect can be zero. It is within this region that oligosaccharides ranging from di- to hexasaccharides (˜400-1500 Da) are frequently located. Although the molecules studied in this work are somewhat larger than this, the NOE effects were still rather weak. Thus, ROESY experiments, which always give positive signals, were used. ROESY permits to assess which protons are close to each other in space, providing key information about the conformation of the molecule. Moreover, these data can also be compared to the results arising from molecular dynamics simulations.

On the other hand, DOSY allows for calculating the diffusion coefficients of the molecules, which in turn gives information about the hydrodynamic radius (volume) that the molecules occupy. While the biological evaluation of the compounds 1-3 was carried out in water, due to their poor water solubility, which would have led to unrealistically long experiment times, the NMR experiments were performed in deuterated methanol. The properties of methanol are rather similar to water with respect to polarity and dielectric constant (p=1.85 D, ε=80.1 and p=1.69 D, ε=32.7 for water and methanol, respectively). Unfortunately, the signals from the three different mannobiose units were practically indistinguishable from each other, indicating that the chemical environments of all the mannobiose moieties are fairly similar. Notably, since ROESY experiments were performed, the lack of correlations can provide as much information as the correlations themselves, since they are less sensitive to molecular motion than the NOESY analogues.

The computational protocol is described in the “general” section. In particular, the geometries obtained through molecular dynamics simulations were used to extract key interproton distances, which were compared to those estimated from the NOESY experiments using well established approaches.

### Results

DOSY was used for estimating the size of the molecules. Rather surprisingly, while the linker lengths of 1-3 vary significantly, the diffusion coefficients are practically identical. Intuitively, it might be expected that the three arms of the molecules would try to spread out as much as possible to minimize steric interactions. However, this does not seem to be the case. Due to the use of a highly polar solvent (deuterated methanol), these nonpolar molecules are rather interacting with themselves, trying to minimize the contact with the solvent and thus forming crumpled structures that fold back on themselves. The severe overlapping in the ROESY spectra makes it impossible to distinguish correlations between different mannobiose units from those arising within one particular entity. Therefore, herein are only reported the observed correlations, safely assuming that for an observed ROE, the corresponding distance cannot be larger than 4 Å. This is the approximate upper limit of distances that produce a NOE effect in molecules of this size.

### Reference Compound 1

For large molecules with many rotatable bonds, the energy minima are not very well defined. This means that in solution, a large number of different conformations will be present. This is true also in this case. During the molecular dynamics simulations a large number of conformations were observed. During the high temperature of the annealing sequence, the dominating conformation for the molecule was one where each arm is pointing straight out, but as the system cooled down the molecule started to fold together. In fact, at low temperatures this was the dominating conformation.

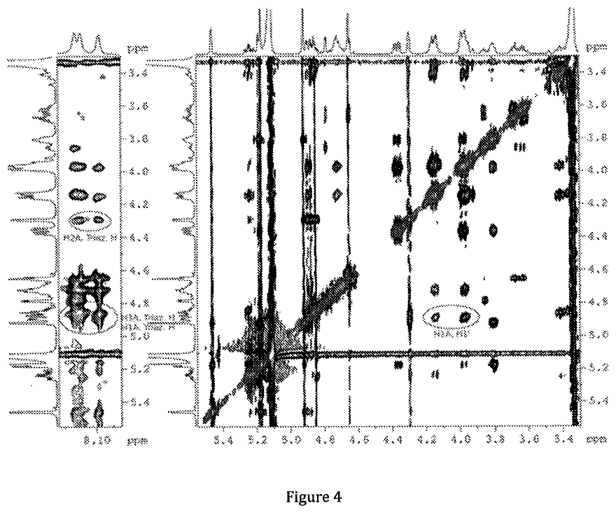

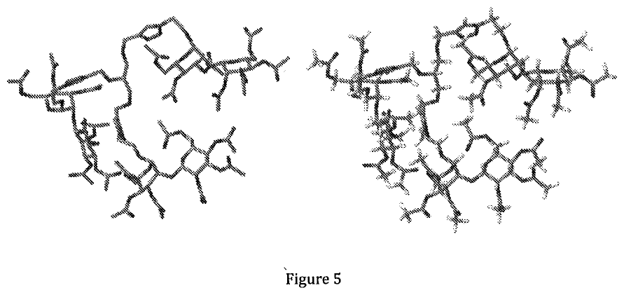

The most important ROESY correlations for this molecule, supporting the folded structure, were the correlations from the triazole and linker protons. The triazole protons showed correlations to the H-2A and H-3A protons and to the anomeric protons H1A (FIG. 4). The distances acquired from the molecular dynamics simulation were 3.3 Å, 3.2 Å and 4.0 Å respectively. Furthermore the H-2B protons showed a correlation to the H-1A protons, which is characteristic for the αβ configuration of the β-(1→2)-linked mannosides present in the molecule. The most important correlations are shown in FIG. 4. In addition, correlations could be seen between protons H3A and H5A as well as H1B, H3B and H5B. This confirmed that the carbohydrate moieties were in ⁴C₁ conformations. The most populated type of conformations for compound 1 during the molecular dynamics simulation, in agreement with the NOE data, is depicted in FIG. 5.

### Reference Compound 2

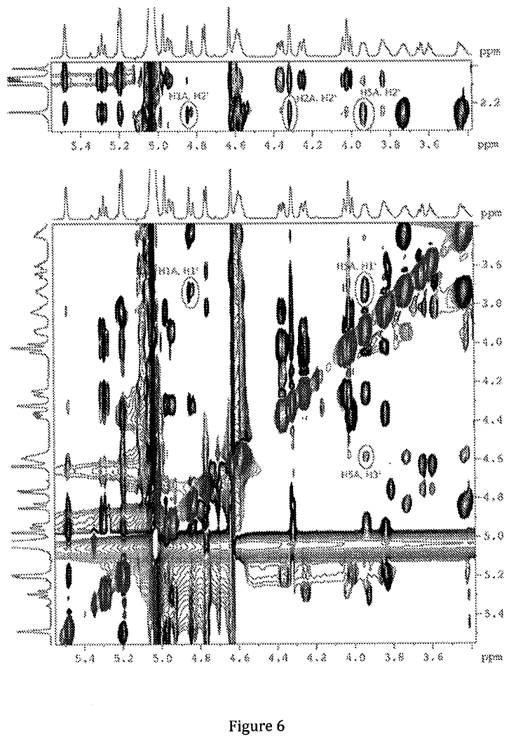

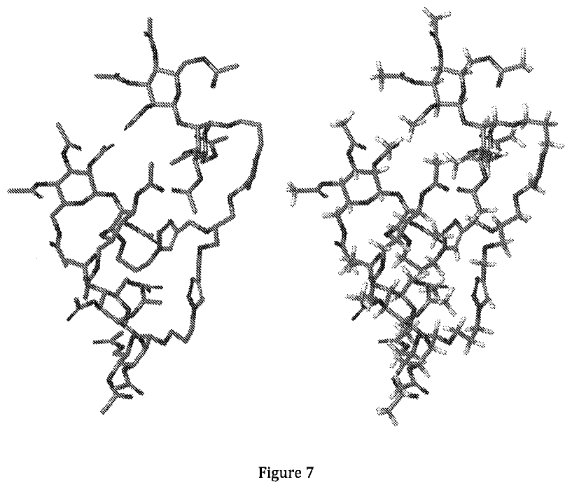

The behavior of the slightly larger compound 2 was very similar to the behavior of compound 1. The main difference in the ROESY spectrum was that the addition of one more —CH₂— moiety located the triazole ring far enough to preclude any ROE correlation to the carbohydrate protons. Since no relevant correlations from the triazole protons could be seen, the conformation was determined by looking at correlations between the linker and carbohydrate protons. The relevant correlations in this case were H1A-H1′ (2.4 Å), H1A-H2′ (4.0 Å), H5A-H1′ (2.6 Å), H5A-H2′ (3.6 Å) and H5A-H3′ (3.6 Å) (FIG. 6). The most populated conformation extracted from the molecular dynamics simulation that agreed with the experimental data is shown in FIG. 7.

### Compound 3

As expected, the behavior of the largest molecule was slightly different from the other two. The linker unit is significantly longer, which allowed for more flexibility and thus an even less well-defined energy minimum compared to the two smaller molecules.

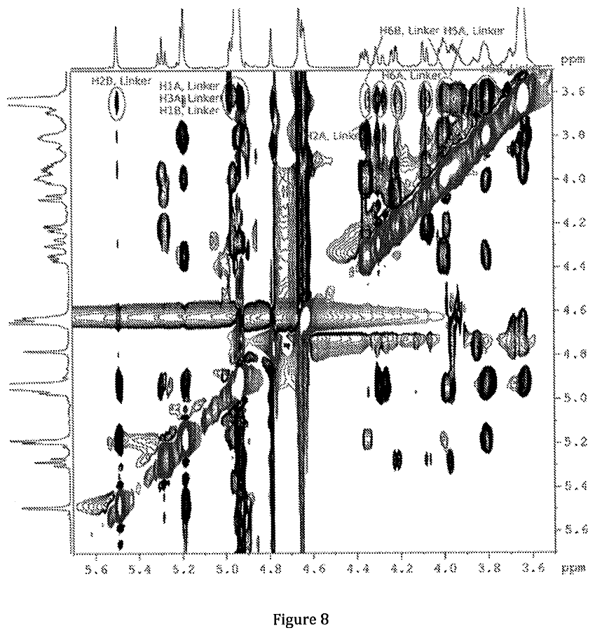

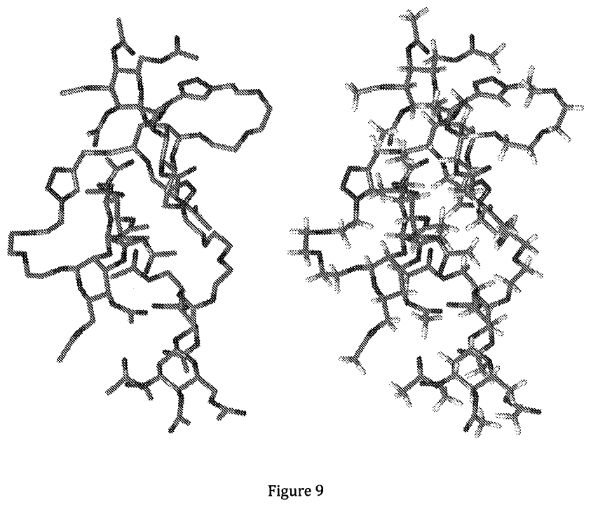

In the ROESY spectrum, correlations from various carbohydrate protons to the linker unit could be seen. This was in accordance with a compact structure such as that displayed in FIG. 9. Notably, there were correlations between the linker protons and almost all sugar protons except H4A and H4B (FIG. 8). This fact should be expected unless the glycosidic linkage between the carbohydrate moieties adopted highly unusual conformations. According to the molecular dynamics simulation the glycosidic linkages were in the expected conformations with the glycosidic ϕ angle being around 60° and the aglyconic ψ angle being around 20-30° consistent with the typical conformation of β-(1→2) is linked mannosides. As in the other two structures, no correlations between the glycerol protons and the carbohydrate or linker protons could be seen. The most populated conformation for compound 3 extracted from the molecular dynamics simulation are shown in FIG. 9.

### CONCLUSIONS

The folded structures of all three compounds leave the triazole moieties on the outside of the molecule. This makes them accessible for biological targets, and it is known that triazoles can take part in biological events. In fact, several pharmaceuticals have been designed around triazole moieties. While in the present case the possible influence of the triazole moiety on the biological activity of the glycoclusters 1, 2, and 3 could not be ruled out completely, it must be emphasized that out of a very large number of oligovalent β-(1→2) mannosides screened, of which several others also contained triazole units, the only compounds showing promising in vitro activities were previously known compounds 1 and 2 and their novel analogue compound 3. Common features shared by these three active compounds are: 1) fully acetylated trivalent β-(1→2) mannobiose units, which 2) via β-linkages are connected to the central core, via 3) linkers of varying sizes. This three-dimensional structure appears to be highly specific and required for biological activity with the corresponding deacetylated or via β-linkages to the linker and central core connected analogues of compound 1 being inactive at least in the in vitro PBMC model. In addition, in all three molecules, the glycerol backbone stands on the opposite side of the sugar moieties, i.e., the glycan moieties are all folded to the same side of the molecule. This is supported by the fact that the protons in the backbone do not show any ROESY correlations to the carbohydrate protons.

It will be obvious to a person skilled in the art that, as the technology advances, the inventive concept can be implemented in various ways. The invention and its embodiments are not limited to the examples described above but may vary within the scope of the claims.

1. A compound of formula (I): 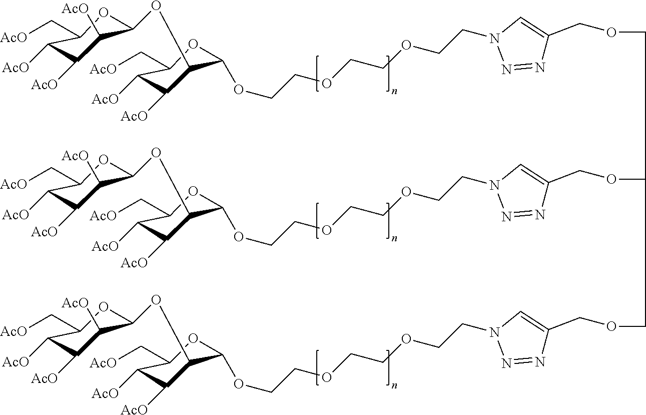
wherein each n is 0 to 2
or a pharmaceutically acceptable salt thereof.

2. A compound as claimed in claim 1 having formula (II): 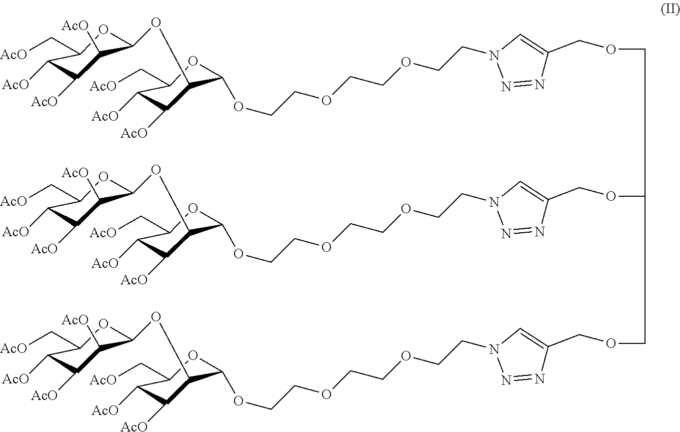
or a pharmaceutically acceptable salt thereof.

3. A compound or a pharmaceutically acceptable salt thereof as claimed in claim 1 configured as a medicament.

4. A compound or a pharmaceutically acceptable salt thereof as claimed in claim 1 configured for therapy.

5. A compound or a pharmaceutically acceptable salt thereof as claimed in claim 1 configured as an adjuvant of a vaccine.

6. A compound or a pharmaceutically acceptable salt thereof as claimed in claim 1 configured for treatment of a condition treatable by Treg and/or Th1-type inducement, and/or Th2-type immune response inhibition.

7. The compound according to claim 6, wherein the condition is type I immediate atopic allergy.

8. The compound according to claim 6, wherein the condition is selected from the group consisting of;
a) atopic eczema/dermatitis syndrome (AEDS),
b) allergic asthma,
c) allergic rhinitis
d) allergic urticaria,
e) food allergy,
f) venom allergy, and
g) allergic rhinoconjunctvitis.

9. The compound according to claim 3, wherein the condition is an infectious disease.

10. The compound according to claim 3, wherein the condition is cancer.

11. An immunostimulatory composition comprising:
one or more compounds according to claim 1; and
a pharmaceutically acceptable carrier, selected from the group consisting of transdermal carriers, transmucosal carriers, oral carriers, parenteral carriers, carriers for depot formulations, and carriers for extended release formulations.

12. The immunostimulatory composition as claimed in claim 11, wherein the carrier is a transmucosal carrier for sublingual and/or buccal administration.

13. The immunostimulatory composition as claimed in claim 11, comprising:
an allergen preparation for specific allergen immunotherapy; and/or
an additional allergy or asthma medicament.

14. The immunostimulatory composition as claimed in claim 11, comprising:
a microbe-specific antigen for vaccination against infectious disease; and/or
an antimicrobial agent.

15. The immunostimulatory composition as claimed in claim 11, comprising:
a cancer antigen for eliciting a specific immune response against cancer cells expressing said antigen; and/or
a cancer medicament.

16. A compound as claimed in claim 1 configured as a food additive or a nutritional preparation.
